(19) United States
(12) Patent Application Publication
(10) Pub. No.: US 2024/0173438 A1
(43) Pub. Date: May 30, 2024

(54) NEAR-INFRARED-II FLUORESCENT COMPOSITE, ITS PREPARATION METHOD AND USES THEREOF

(71) Applicant: Chung Yuan Christian University, Taoyuan City (TW)

(72) Inventors: Cheng-An LIN, Taoyuan City (TW); Yi-Tang SUN, New Taipei City (TW); Min-Hua CHEN, Taoyuan City (TW)

(73) Assignee: Chung Yuan Christian University, Taoyuan City (TW)

(21) Appl. No.: 18/071,647

(22) Filed: Nov. 30, 2022

(51) Int. Cl. A61K 49/00; C09K 11/02; C09K 11/58

(52) U.S. Cl. CPC A61K 49/0065; C09K 11/025; C09K 11/58; B82Y 20/00

(57) ABSTRACT

Disclosed herein are second near-infrared (NIR-II) fluorescent composite and its production method. The method mainly includes the steps of, mixing a gold nanocluster having a plurality of a thiol-based compound on its outer surface and alpha-glycerylphosphorylcholine (alpha-GPC) in a solvent to form a mixture; replacing the solvent with an inert gas; and heating the mixture at a temperature about 100-200° C. in the presence of the inert gas until at least a portion of the gold nanocluster is encapsulated by a capping layer consisting of alpha-GPC, thereby producing the NIR-II fluorescent composite. The thus-produced NIR-II fluorescent composite is characterized by having an emission wavelength covering NIR-II region detectable by specialized camera. Also encompassed in the present disclosure is a method for conducting in vivo bioimaging of a target area in a subject. The method includes administering the present NIR-II fluorescent composite to the target area; and detecting the fluorescence emitted therefrom at a wavelength between 900 to 1700 nm.

### BACKGROUND OF THE INVENTION

### 1. Field of the Invention

The present disclosure relates to a second near-infrared (NIR-II) fluorescent composite. More particularly, the disclosed invention relates to the NIR-II fluorescent composite for use as a luminous material, which is useful as a developer or contrast agent for intravital microscopy or diagnostic imaging.

### 2. Description of Related Art

Imaging technologies such as magnetic resonance imaging (MRI), computed tomography (CT), positron emission topography (PET), X-ray radiography, and fluoroscopy are clinically routine procedures for visualizing abnormalities in a tissue image with the aid of a contrast agent or developer. Contrast agents or developers interact with the incident radiation to produce visible changes in the acquired image, thus may increase the sensitivity of the imaging techniques in identifying pathologies that are previously undetectable.

Real-time X-ray radiography and near-infrared (NIR) imaging are often employed during surgery to help surgeons visualize the lesion. However, the use of X-ray radiography is limited by the maximum radiation dose the clinical staff may be exposed to. As to NIR imaging, the widely used contrast agent—indocyanine green (ICG), possessing the first near-infrared (NIR-I) fluorescence, is unstable and sensitive to solvents, concentrations and excitation conditions, thus, ICG has a short half-life and limited injection depth.

In view of the foregoing, there exists in the related art a need of an improved and stable NIR-II fluorescent materials covering the wavelengths from 900 nm to 1700 nm for imaging a target area that is within the deeper part of body.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to near-infrared-II (NIR-II) fluorescent composite comprising a gold nanocluster and a capping layer. The gold nanocluster has a plurality of thiol-based compounds on its outer surface; and the capping layer consists of alpha-glycerylphosphorylcholine (alpha-GPC) and encapsulates at least a portion of the gold nanocluster; wherein the NIR-II fluorescent composite has an emission wavelength between 900 to 1700 nm

According to some embodiments of the present disclosure, the gold nanocluster is about 1 to 3 nm in diameter.

According to some embodiments of the present disclosure, the thiol-based compounds are dihydrolipoic acids (DHLAs), glutathiones, mercaptobenzoic acids (MBAs), poly(ethylene glycol) dithiols, methoxy polyethylene glycol thiols (mPEG thiols), or a combination thereof.

Another aspect of the present disclosure is directed to a method for producing a near-infrared-II (NIR-II) fluorescent composite. The method comprises steps of (a) mixing a gold nanocluster and alpha-glycerylphosphorylcholine (alpha-GPC) in a solvent to form a first mixture, wherein the gold nanocluster has a plurality of thiol-based compounds on its outer surface; (b) replacing the solvent in the first mixture with an inert gas thereby forming a second mixture; and (c) heating the second mixture at a temperature about 100-200° C. until at least a portion of the gold nanocluster is encapsulated by a capping layer consisting of GPC, thereby producing the NIR-II fluorescent composite.

According to some embodiments of the present disclosure, the thiol-based compounds are dihydrolipoic acids (DHLAs), glutathiones, mercaptobenzoic acids (MBAs), poly(ethylene glycol) dithiols, methoxy polyethylene glycol thiols (mPEG thiols), or a combination thereof.

According to some embodiments of the present disclosure, the second mixture in step (c) of the present method is heated at about 160° C. for about 10 to 60 minutes in the presence of nitrogen.

According to some embodiments of the present disclosure, the solvent is selected from the group consisting of ethanol, methanol, propanol, butanol, pentanol, hexanol, heptanol, octanol and a combination thereof.

Still another aspect of the present disclosure is directed to a method for bioimaging of a target area in a subject. The method comprises at least steps of (a) administering an effective amount of the aforementioned NIR-II fluorescent composite to the target area; and (b) detecting the fluorescence emitted from the aforementioned NIR-II fluorescent composite in the target area at a wavelength between 900 to 1700 nm.

According to some embodiments of the present disclosure, the target area is a normal tissue, a malignant tissue, or a combination thereof.

According to some embodiments of the present disclosure, the present NIR-II fluorescent composite is administered to the subject in the amount of about 0.01 to 300 mg/kg.

According to some embodiments of the present disclosure, the subject is a mammal. Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A is a line graph depicting the fluorescent intensities and emission wavelengths of the present NIR-II fluorescent composite (AuNCs@DHLA@GPC) according to one embodiment of the present disclosure;

FIG. 1B is a line graph depicting the fluorescent intensities and emission wavelengths of the present NIR-II fluorescent composite (AuNCs@MBA@GPC) according to another embodiment of the present disclosure;

FIG. 2A is a serial short-wave infrared (SWIR) images of a healthy mouse treated with the present NIR-II fluorescent composite according to one embodiment of the present disclosure;

FIG. 2B is a SWIR image of the healthy mouse after 120 seconds of the present NIR-II fluorescent composite treatment according to one embodiment of the present disclosure; and

FIG. 3 is a serial SWIR images of squamous cell carcinoma (SCC) bearing mouse treated with the present NIR-II fluorescent composite according to one embodiment of the present disclosure.

In accordance with common practice, the various described features/elements are not drawn to scale but instead are drawn to best illustrate specific features/elements relevant to the present invention.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. DEFINITIONS

For convenience, certain terms employed in the specification, examples and appended claims are collected here. Unless defined otherwise, all technical and scientific terms used herein have the same meaning as commonly understood by one of the ordinary skilled in the art to which this invention belongs.

The singular forms “a”, “and”, and “the” are used herein to include plural referents unless the context clearly dictates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

The term “second near-infrared” or “near-infrared-II (NIR-II)” is used interchangeably herein and is intended to mean a light spectrum encompassing wavelengths ranging from 900 to 1,700 nm.

The term “composite” as used herein is intended to mean a material formed by the combination of two or more constituents, and the material has properties significantly different from either constituent. According to the present disclosure, a metal cluster and a choline compound are combined to form a composite with a luminescent property differs from that of the metal cluster.

The term “gold nanocluster(s)” or “AuNC(s)” as used herein follows the definition by skilled person in the art and refers to a self-assembly of dozens of gold atoms, in which the assembled gold atoms as a whole has a total diameter of less than 3 nm. Typically, gold nanoclusters absorb light in the near-infrared (NIR) range between 650 and 900 nm and exhibit native luminescent properties that are well-explored in related arts. Note that the gold nanocluster(s) can be further modified with functional groups by any means and procedures well known in the art. According to the present disclosure, the gold nanocluster is modified by thiol-based compounds.

The term “thiol-based compound(s)” as used herein refers to any organosulfur compound comprising one or more functional groups of R—SH, where R represents organic substituents including but not limited to alkyl, alkenyl, alkynyl, and aryl groups. In some embodiments of the present disclosure, the thiol-based compound has one —SH group, whereas in other embodiments of the present disclosure, the thiol-based compound has two —SH groups.

The term “malignant” tissue as used herein is intended to describe any tissue or organ that is cancerous and/or tending to invade normal tissue or to recur after removal. In some embodiments of the present disclosure, the malignant tissue is a squamous cell carcinoma (SCC) tumor.

The term “subject” or “patient” is used interchangeably herein and is intended to mean a mammal including the human that is treatable by the near-infrared-II (NIR-II) fluorescent composite of the present invention. The term “mammal” refers to all members of the class Mammalia, including humans, primates (e.g., monkey, and chimpanzee), domestic and farm animals, such as rabbit, pig, goat, sheep, and cattle; as well as zoo, sports or pet animals (e.g., a horse, a dog, a cat and etc); and rodents, such as mouse, rat, guinea pig, and hamster. In a working example, the subject is a mouse; in another working example, the subject is a human. Further, the term “subject” or “patient” intended to refer to both the male and female gender unless one gender is specifically indicated.

### 2. DETAIL DESCRIPTION OF PREFERRED EMBODIMENTS

The present disclosure is based, at least in part, on the discovery that the emission wavelength of a gold nanocluster (AuNC) red-shifts to second near infrared wavelengths of 900 to 1,700 nm when the gold nanocluster is encapsulated by a capping layer composed of alpha-glycerylphosphorylcholine (alpha-GPC). Hence, the composite made of AuNC and alpha-GPC may serve as a contrast agent for in vivo bioimaging under short-wave infrared image camera. Also disclosed herein is a method for the production of the present near-infrared-II (NIR-II) fluorescent composite.

### 2.1 Near-Infrared-II (NIR-II) Fluorescent Composite and its Preparation Methods

The present disclosure aims at providing a near-infrared-II (NIR-II) fluorescent composite, which comprises in its structure, a gold nanocluster (AuNC) having a plurality of thiol-based compounds on its outer surface; and a capping layer consisting of alpha-glycerylphosphorylcholine (alpha-GPC), wherein the capping layer encapsulates at least a portion of the gold nanocluster. According to embodiments of the present disclosure, the present NIR-II fluorescent composite is produced by,
(a) mixing a gold nanocluster (AuNC) and alpha-glycerylphosphorylcholine (alpha-GPC) in a solvent to form a first mixture, wherein the gold nanocluster has a plurality of thiol-based compounds on its outer surface;
(b) replacing the solvent in the first mixture with an inert gas thereby forming a second mixture; and
(c) heating the second mixture at a temperature about 100-200° C. gas until at least a portion of the gold nanocluster is encapsulated by a capping layer consisting of GPC, thereby producing the present NIR-II fluorescent composite.

The gold nanocluster suitable for use in the present disclosure is an aggregate of dozens to hundreds of gold (Au) atoms self-assembled into a nanocluster in a suitable condition, and the self-assembled nanocluster is then further subjected to surface modification, so that the gold nanocluster has a plurality of thiol-based compounds on its outer surface.

According to embodiments of the present disclosure, the gold nanocluster is made of tens to hundreds of gold atoms, such as 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 gold atoms. Typically, the gold nanocluster formed via self-assembled gold atoms has a core diameter (i.e., the diameter of aggregate gold atoms) less than 3 nm, preferably ranging from 1 to 3 nm. The gold nanocluster is further thiolated by organosulfur compounds (i.e., thiol-based compounds) by methods well known in the art, including “one-pot” synthesis, ligand exchange processes, and the like, in which sulfur-gold (S—Au) bonds are formed via the interaction between organosulfur compounds and the outer surface of the aggregated gold atoms. In some embodiments of the present disclosure, the gold nanocluster for use in the present disclosure is prepared by mixing gold atoms and thiol-based compounds at a molar ratio of about 10:1 to 1:10, for example, about 10:1, 9:1, 8:1, 7:1, 6:1, 5:1, 4:1, 3:1, 2:1, 1:1, 1:2, 1:3, 1:4, 1:5, 1:6, 1; 7, 1:8, 1:9, or 1:10, to form a homogeneous solution, which was kept at 20 to 40° C. for a period of about 10 to 80 hours, until gold atoms self-assemble into a cluster (or an aggregate) with the thiol-based compounds attached to the outer surface of the aggregated gold atoms (i.e., the thus produced gold nanocluster has a plurality of thiol-compounds on its outer surface). Alternatively or optionally, the gold atoms (i.e., typically are gold(III) precursors) are reduced by reducing agent such as sodium borohydride or carbon monoxide, before self-assembling, or at the same time with thiol-based compounds attachment.

Example of thiol-based compound suitable for use in the present disclosure includes but is not limited to dihydrolipoic acid (DHLA), glutathione, mercaptobenzoic acid (MBA), poly(ethylene glycol) dithiol, methoxy polyethylene glycol thiol (mPEG thiol), or a combination thereof. In some preferred examples, the gold nanocluster is formed by mixing α-lipoic acid and chloroauric acid (HAuCl₄) at a molar ratio of 1:3 to 3:1, such that the gold nanocluster thus produced has DHLA coated on its surface. In other examples, the gold nanocluster has DHLA on its outer surface, which is formed by mixing α-lipoic acid and chloroauric acid (HAuCl₄) at a molar ratio of 1:1.7 to 1:2. In still other examples, the gold nanocluster is formed by mixing mercaptobenzoic acid (MBA) and chloroauric acid (HAuCl₄) at a molar ratio of about 1:1 to 3:1, thereby producing the gold nanocluster having MBA on its outer surface.

Once the gold nanocluster is formed, it is mixed with alpha-GPC in a solvent thereby forming a first mixture (step (a)). According to embodiments of the present disclosure, the gold nanocluster and alpha-GPC are mixed at a mass ratio of about 10:1 to 1:10 in a solvent. Examples of the solvent suitable for use in the present step include, but are not limited to, ethanol, methanol, propanol, butanol, pentanol, hexanol, heptanol, octanol, and a combination thereof. According to preferred embodiments of the present disclosure, the gold nanocluster and alpha-GPC are mixed at the mass ratio of about 1:10 in methanol.

Once the gold nanocluster and alpha-GPC are fully mixed in the solvent, the solvent is replaced with an inert gas. Specifically, the solvent (e.g., methanol) in the first mixture is removed by evacuator, and the reaction flask is then filled with nitrogen thereby forming a second mixture, which is heated at a temperature about 100-200° C. until at least a portion of the gold nanocluster is encapsulated by a capping layer consisted of alpha-GPC (steps (b) and (c)), thereby producing the present NIR-II fluorescent composite. In other words, the reaction (step (c)) is conducted under the inert atmosphere. According to some embodiments of the present disclosure, the capping layer encapsulates or covers a portion of, or the entire outer surface of the gold nanocluster via forming chemical bonds between alpha-GPC and thiol-based compounds disposed on the outer surface of the gold nanocluster, or via alpha-GPC doping between the thiol-based compounds. In some working examples of the present disclosure, the second mixture is heated at about 160° C. for about 10 to 60 minutes in the presence of nitrogen, thereby produce a solid product, i.e., the present NIR-II fluorescent composite.

In some alternative or optional embodiments, the solid product can be resuspended in aqueous buffer (e.g., phosphate buffered saline, PBS) for storage and subsequent use.

According to the present disclosure, the thus produced NIR-II fluorescent composite has an emission wavelength ranging from 750 to 1,700 nm, such as 750, 800, 850, 900, 950, 1000, 1050, 1100, 1150, 1200, 1250, 1300, 1350, 1400, 1450, 1500, 1550, 1600, 1650, and 1700 nm; preferably from 900 to 1,400 nm, such as 900, 950, 1000, 1050, 1100, 1150, 1200, 1250, 1300, 1350, and 1400 nm. In some preferred embodiments, the NIR-II fluorescent composite has a peak emission at about 700 nm. In other examples, the present NIR-II fluorescent composite has a peak emission at about 900 nm. In further examples, the present NIR-II fluorescent composite has a peak emission at about 1,050 nm.

### 2.2 Methods for Conducting Bioimaging In Vivo

The present disclosure also aims at providing a novel bioimaging method for non-invasively visualizing a biological activity in live subject in real time. To this purpose, the present NIR-II fluorescent composite may be applied to a target area of a subject and serves as a developer and/or contrast agent to improve resolution and retention time in deep tissues. The present disclosure thus encompasses a method for conducting in vivo bioimaging of a target area in a subject.

In some embodiments, the method comprises administering an effective amount of the present NIR-II fluorescent composite to the target area of the subject; and detecting the fluorescence emitted from the present NIR-II fluorescent composite in the target area at a wavelength between 900 to 1700 nm.

According to the present disclosure, the present NIR-II fluorescent composite is formulated into formulations such as powders, suspensions, solutions, or gels that can be manufactured by any method known in the art of pharmacology, by using inert, essentially nontoxic, pharmaceutically suitable carriers or solvents. Relative amounts of the NIR-II fluorescent composite in a formulation described herein will vary, depending upon the position and/or size of the target area, and/or condition of the subject, and further depending upon the route by which the composition is to be administered.

According to the present disclosure, the target area can be a normal tissue, a malignant tissue, or a combination thereof. In some embodiments, the present NIR-II fluorescent composite is manufactured as a solution form therefore is administered to the circulatory system and/or tumors of a subject.

According to some embodiments of the present disclosure, the NIR-II fluorescent composite is administered to the target area of the subject in an amount sufficient to retain in the subject for a period of time. According to some embodiments of the present disclosure, the present NIR-II fluorescent composite is administered to the target area in the amount of 0.01 to 100 mg/kg; for example, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 10, 15, 20, 25, 30, 35, 40, 45, 50, 55, 60, 65, 70, 75, 80, 85, 90, 95, or 100 mg/kg. In one specific example of the present disclosure, the present NIR-II fluorescent composite is administered to the subject in the amount of about 1.6 mg/kg. In another specific example, the present NIR-II fluorescent composite is administered to the subject in the amount of about 20 mg/kg.

In some embodiments, the subject is a mammal, preferably is a human.

By the virtue of the above features, the present method can provide a novel contrast agent that do possess the second-near infrared emission in target tissues detectable by specialized camera, thereby allowing a rapid, potent, and efficient bioimaging in vivo.

### EXAMPLES

### Materials and Methods

### I. Gold Nanoclusters (AuNCs) Synthesis

### 1. AuNCs@DHLA

Chloroauric acid (HAuCl₄, 2.5 mM, 4.854 mL) was added into α-lipoic acid (1.45 mM, dissolved in 20 mL of deionized water) solution and the total volume was brought to 465 mL. The mixture was stirred at 300 rpm for 5 minutes, then 9.5 mL of 50 mM NaBH₄ was added dropwise. The mixture was stirred for 15 hours at room temperature and then concentrated to about 10 mL via vacuum evaporation at 40° C. The concentrated mixture was then filtered (membrane filter with a cutoff molecular weight of 10 kDa) and the filtrate was centrifuged at 3,500 rpm for 13 minutes. The resulting filtrate containing gold nanoclusters (AuNCs) having dihydrolipoic acid (DHLA) coated thereon were stored at room temperature until use.

Alternatively, AuNCs@DHLA was produced by steps as follows: chloroauric acid (HAuCl₄, 2.5 mM, 10 mL) was added into α-lipoic acid (1.45 mM, dissolved in 10 mL of deionized water) and the total volume of the solution was brought to 465 mL. The mixture was stirred at 300 rpm for 5 minutes, then 20 mL of 50 mM NaBH₄ was added dropwise. The mixture was stirred for 15 hours at room temperature and then concentrated to about 10 mL via vacuum evaporation at 40° C. The concentrated mixture was then filtered (membrane filter with a cutoff molecular weight of 10 kDa) and the filtrate was centrifuged at 3,500 rpm for 13 minutes. The resulting filtrate containing gold nanoclusters (AuNCs) having dihydrolipoic acid (DHLA) coated thereon (concentration: 80 μM) were stored at room temperature until use.

### 2. AuNCs@MBA

Mercaptobenzoic acid (MBA, 0.77 g dissolved in 10 mL of 150 mM NaOH) and HAuCl₄ (50 mM, 9.709 mL) were mixed into 238.75 mL of H₂O. The mixture was stirred at 300 rpm for 5 minutes, then 200 μL of 1M NaOH was added, followed by addition of carbon monoxide (CO) for 2 minutes. The mixture was stirred for 3 days at room temperature and then concentrated to about 10 mL via vacuum evaporation at 40° C. The concentrated mixture was then filtered (membrane filter with a cutoff molecular weight of 10 kDa) and the filtrate was centrifuged at 3,500 rpm. The filtration and centrifugation repeated four times. The resulting filtrate containing gold nanoclusters (AuNCs) having MBA coated thereon were stored at room temperature.

### II. NIR-II Fluorescent Composite Preparation

AuNCs@DHLA or AuNCs@MBA (1 mL) was placed in a round-bottom flask, liquid was removed by evacuator, followed by adding 10 g of alpha-glycerylphosphorylcholine (alpha-GPC) powder into the flask. Methanol (20 mL) was added into the flask until the gold nanoclusters and alpha-GPC were fully mixed. The methanol in the flask was removed by evacuator, and the flask was filled with nitrogen at a constant temperature of 160° C. for 5 to 45 minutes to form a colloid, which was cooled to room temperature, thereby producing the NIR-II fluorescent composite. The colloid was re-dissolved in PBS, and the solution was subjected to centrifugation (at 3500 rpm) and filtration (using a membrane filter having a cutoff molecular weight at 10 kDa) for three times, thereby producing a final product that could be subjected to sterilization and stored at ambient conditions.

### PEGylation and Protein Adsorption Characterization

The present NIR-II fluorescent composite (10 μL) was mixed with 10 μL of mPEG₅₅₀-NH₂ (MW: 550, 1 mM), and serial diluted by 1-Ethyl-3-(3-dimethylaminopropyl)carbodiimide (EDC) from 512 mM to 0.23 mM. The mixture was reacted at room temperature overnight, such that the NIR-II fluorescent composite was PEGylated and cross-linked. The optimal EDC concentration was determined by gel electrophoresis (2%). The PEGylated NIR-II fluorescent composite was incubated in fresh bovine serum albumin (BSA) solution (10 mM) at 37° C. with shaking at 200 rpm for 2 h. The BSA-adsorbed PEGylated NIR-II fluorescent composite was recovered after ultra-centrifugation and washed with 4×1 mL PBS to remove free BSA. The ability of BSA absorption of the PEGylated NIR-II fluorescent composite was verified by gel electrophoresis (2%) and detected by short wave infrared (SWIR) camera with a long-pass filter of 1,070 nm.

### Animal

Male ICR mice (4 to 6 weeks ages) were obtained from National Laboratory Animal Center (Taipei) and housed in a polycarbonate cage (cage size of 33.2 cm×21.5 cm×21 cm) in well-ventilated room at the temperature of 18-26° C. and a relative humidity of 30%-70% with ad libitum access to food and water. Normal illumination was provided and maintaining 12-hour light/dark cycle.

### Xenograft Squamous Cell Carcinoma (SCC) Animal Model

Each mouse (7-8 weeks old) was injected with 2×10⁵ squamous cells subcutaneously to generate SCC on day 0, and then returned to culture for another 14 days to allow tumor progression. To evaluate the efficacy of the present NIR-II fluorescent composite, each mouse was placed under SWIR camera with 1070 nm filter, and received one dose of the composite (0.4 mg) through tail vein injection. The circulation of the present NIR-II fluorescent composite in each mouse was video recorded, and single fluorescent images were individually captured at 0, 5, 10, 15 and 20 seconds. For comparison, the healthy control mice (i.e., mice without xenografted SCC on their backs) received the same treatment of the present NIR-II fluorescent composite, and single fluorescent images were individually captured at 0, 3, 10, 30 and 120 seconds.

### Example 1 Characterization of the Present NIR-II Fluorescent Composite

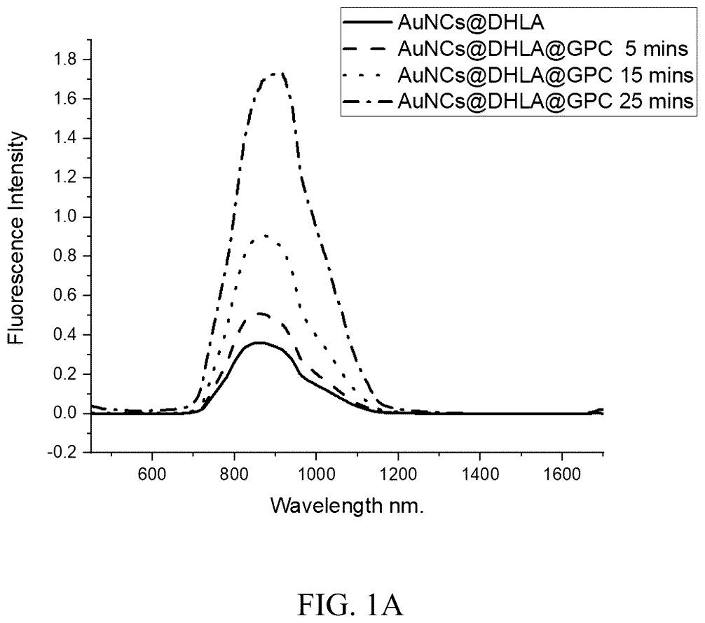

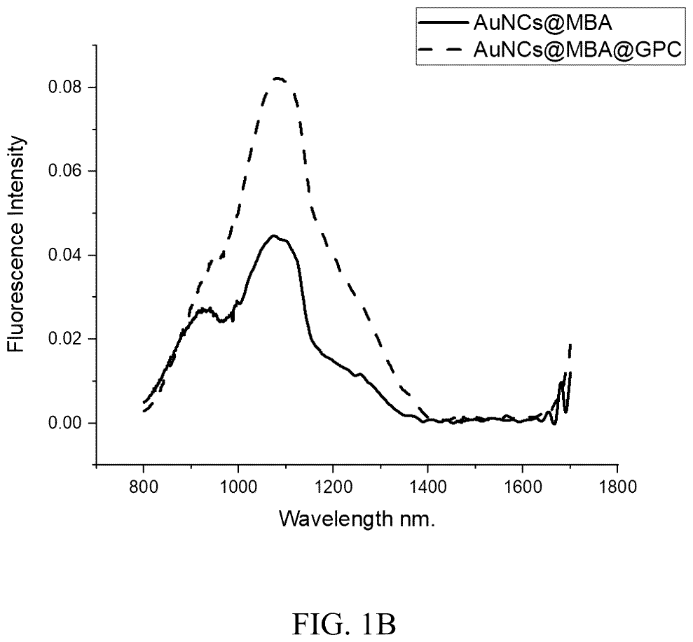

The present NIR-II fluorescent composite was produced via use of either AuNCs@DHLA or AuNCs@MBA as the core structure (hereinafter, AuNCs@DHLA@GPC and AuNCs@MBA@GPC) according to the method described in the “Materials and Methods” section. In this example, whether the emissions of AuNCs@DHLA@GPC and AuNCs@MBA@GPC shifted to second near-infrared region were examined by SWIR camera, and results are shown in FIGS. 1A to 1B.

It was found that, when excited by the light of 750 nm, the emission peak of the present AuNCs@DHLA@GPC shifted from 850 nm to 950 nm, and the emission wavelength ranged from 700 to 1700 nm. The color of AuNCs@DHLA@GPC composite observed under ultraviolet light changed from orange to deep red (data not shown). Further, the emission intensity increased with the increase of the reaction time. As to AuNCs@MBA@GPC composite, it also exhibited an emission peak of 1050 nm when excited by the light of 750 nm, and the emission wavelength ranged from 700 to 1400 nm. Data as depicted in FIGS. 1A and 1B collectively indicated that the present NIR-II fluorescent composite could emit lights having wavelengths encompassing the first and second near-infrared regions.

### Example 2 Stability of the Present NIR-IT Fluorescent Composite

In this example, the stability of the present NIR-II fluorescent composite was investigated. To this purpose, the outer surface of the present NIR-II fluorescent composite was PEGylated, and then the PEGylated or non-PEGylated NIR-II fluorescent composites were respectively mixed with BSA protein. The amount of BSA protein adsorbed on the surface of the PEGylated or non-PEGylated NIR-II fluorescent composite was determined in accordance with the procedure described in “Materials and Methods” section of this paper.

It was found that, BSA protein was adsorbed on the surface of the non-PEGylated NIR-II fluorescent composite but not on that of the PEGylated NIR-II fluorescent composite, indicating that the NIR-II fluorescent composite was amenable to PEGylation to prevent protein adsorption. Meanwhile, the fluorescence emitted from both non-PEGylated and PEGylated NIR-II fluorescent composites could be successfully detected by SWIR camera with 1070 nm long-pass filter when being excited by the light of 780 nm, indicating that PEGylation to the present NIR-II fluorescent composite did not affect its luminous characteristics. The data collectively indicated that the present NIR-II fluorescent composites were amenable to surface modification and capable of keeping the stability of luminous efficacy.

### Example 3 In Vivo Imaging Using the Present NIR-II Fluorescent Composite

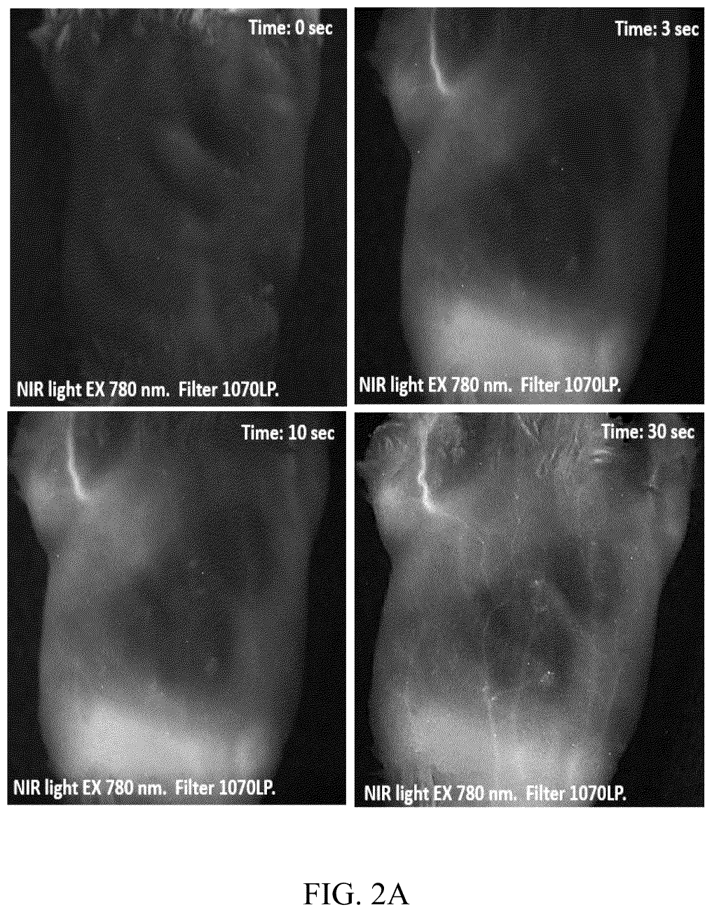

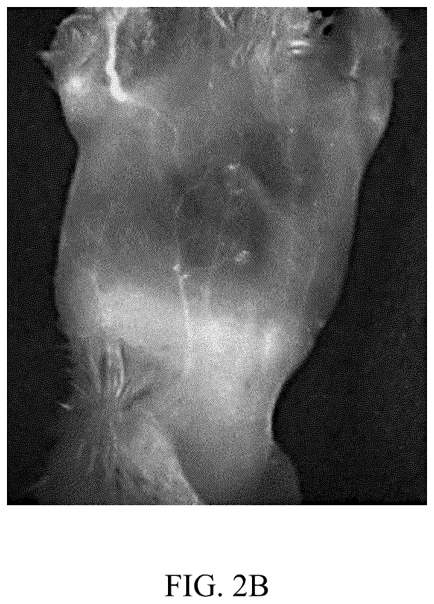

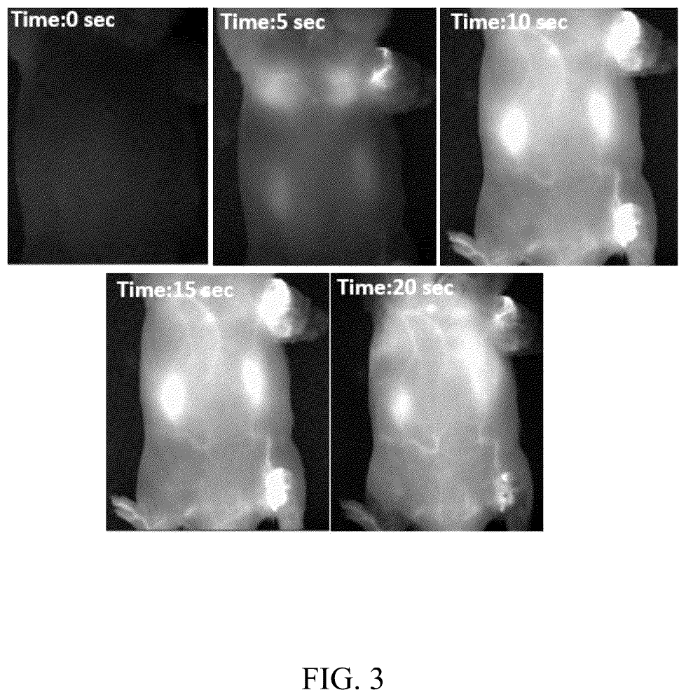

In this example, the present NIR-II fluorescent composite was administered to healthy mice and mice having xenografted squamous cell carcinoma (SCC) on their back and images were captured at designated time in accordance with procedures described in the “Materials and Methods” section, and results are provided in FIGS. 2 to 3.

Referring to FIGS. 2A and 2B, which are images of a healthy mouse injected with low dose (0.4 mg per mouse) of the present NIR-II fluorescent composite, the images were respectively captured at 0, 3, 10, and 30 seconds after the injection (FIG. 2A), and the fluorescent intensity was found to last for 120 seconds (FIG. 2B).

As for SCC-bearing mice, it was found that the present NIR-II fluorescent composite started to accumulate in tumor area 10 seconds after the injection, and the enhanced permeability and retention (EPR) effect lasted at least 20 seconds (FIG. 3).

Taken together, the data of Examples 1 to 3 collectively indicates that the present NIR-II fluorescent composite does not only possess a broad emission covering NIR-I and NIR-II, but also exhibit improved, stable second-near-infrared illumination in live subjects. Hence, the present NIR-II fluorescent composite could serve as a novel contrast agent for in vivo bioimaging.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A near-infrared-II (NIR-II) fluorescent composite, comprising:
a gold nanocluster having a plurality of thiol-based compounds on its outer surface; and
a capping layer consisting of alpha-glycerylphosphorylcholine (alpha-GPC) and encapsulating at least a portion of the gold nanocluster;
wherein, the NIR-II fluorescent composite has an emission wavelength between 900 to 1700 nm.

2. The NIR-II fluorescent composite of claim 1, wherein the gold nanocluster is about 1 to 3 nm in diameter.

3. The NIR-II fluorescent composite of claim 1, wherein the thiol-based compounds are dihydrolipoic acids (DHLAs), glutathiones, mercaptobenzoic acids (MBAs), poly(ethylene glycol) dithiols, methoxy polyethylene glycol thiols (mPEG thiols), or a combination thereof.

4. A method for producing a near-infrared-II (NIR-II) fluorescent composite, comprising:
(a) mixing a gold nanocluster and alpha-glycerylphosphorylcholine (alpha-GPC) in a solvent to form a first mixture, wherein the gold nanocluster has a plurality of thiol-based compounds on its outer surface;
(b) replacing the solvent in the first mixture with an inert gas thereby forming a second mixture; and
(c) heating the second mixture at a temperature about 100-200° C. until at least a portion of the gold nanocluster is encapsulated by a capping layer consisting of alpha-GPC, thereby producing the NIR-II fluorescent composite.

5. The method of claim 4, wherein the thiol-based compounds are dihydrolipoic acids (DHLAs), glutathiones, mercaptobenzoic acids (MBAs), poly(ethylene glycol) dithiols, methoxy polyethylene glycol thiols (mPEG thiols), or a combination thereof.

6. The method of claim 4, wherein in step (c), the second mixture is heated at about 160° C. for about 10 to 60 minutes in the presence of nitrogen.

7. The method of claim 4, wherein the solvent is selected from the group consisting of ethanol, methanol, propanol, butanol, pentanol, hexanol, heptanol, octanol and a combination thereof.

8. A method for bioimaging of a target area in a subject, comprising,
(a) administering an effective amount of the NIR-II fluorescent composite of claim 1 to the target area; and
(b) detecting the fluorescence emitted from the NIR-II fluorescent composite of claim 1 in the target area at a wavelength between 900 to 1700 nm.

9. The method of claim 8, wherein the target area is a normal tissue, a malignant tissue, or a combination thereof.

10. The method of claim 8, wherein the NIR-II fluorescent composite is administered to the subject in the amount of about 0.01 to 300 mg/kg.

11. The method of claim 8, wherein the subject is a mammal.

12. The method of claim 11, wherein the mammal is a human.
